(19) United States
(12) Patent Application Publication
(10) Pub. No.: US 2026/0248473 A1
(43) Pub. Date: Aug. 27, 2026

(54) X-RAY CT DEVICE, IMAGE CAPTURE METHOD, AND PROGRAM

(71) Applicant: Sapporo Medical University, Hokkaido (JP)

(72) Inventors: Yoshiya OHASHI, Hokkaido (JP); Keishi OGURA, Hokkaido (JP)

(21) Appl. No.: 19/139,119

(22) PCT Filed: Dec. 13, 2023
(86) PCT No.: PCT/JP2023/044588

§ 371 Date: Jun. 13, 2025

(30) Foreign Application Priority Data
Dec. 14, 2022 (JP) 2022-199377

(51) Int. Cl. A61B 6/50; A61B 6/03

(52) U.S. Cl. CPC A61B 6/504; A61B 6/032

(57) ABSTRACT

The X-ray CT device includes: a main scanner (154) that captures a CT image of a diagnostic area at a timing when a contrast agent is present in an artery being monitored; and a monitoring scanner (153) that monitors whether a CT value in a vein monitoring area obtained by repeatedly emitting an X-ray from an X-ray source toward a subject is greater than or equal to a first threshold value after the main scanner (154) finishes capturing a CT image of the diagnostic area. The main scanner (154) captures a CT image of the diagnostic area when the monitoring scanner (153) determines that a CT value in the vein monitoring area is greater than or equal to a first threshold value.

### TECHNICAL FIELD

The present disclosure relates to an X-ray CT device, an image capture method, and a program.

### BACKGROUND ART

Computed tomography (CT) angiography is widely performed in which a contrast agent is injected into a body of a subject and an X-ray CT device photographs a diagnostic area that is an area to be diagnosed of the subject. In CT angiography, in order to obtain a clear image of a diagnostic area without increasing the amount of a contrast agent administered to a subject or the number of times of photographing, the diagnostic area needs to be photographed at a timing when the contrast agent reaches the diagnostic area.

For example, Patent Literature 1 discloses a method of continuously photographing an aorta at an interval of 1 second after injection of a contrast agent and estimating a peak time of concentration of the contrast agent. Patent Literature 1 does not specifically mention a technique for photographing a vein, and it can be considered that a general approach is used, that is, a delay time is set with reference to when an artery is photographed and a vein is photographed at a point in time when the delay time elapses.

### CITATION LIST

### Patent Literature

Patent Literature 1: Unexamined Japanese Patent Application Publication No. 2019-180444

### SUMMARY OF INVENTION

### Technical Problem

In recent years, due to spread of minimally invasive surgery using a laparoscope or a surgical support robot, there is a demand for understanding a position and a shape of a vein in detail before surgery. For example, in transverse colon cancer surgery, there is a demand for checking a clearly captured three-dimensional CT image of an area around a superior mesenteric vein before surgery in order to avoid laceration of a fragile vein. However, the method disclosed in Patent Literature 1 has difficulty in clearly photographing a vein for each subject, because a contrast agent injected into a body is diluted as circulating throughout the body from a heart and time required for reaching a vein of a diagnostic area differs for each subject.

The present disclosure has been made based on such a background, and an objective of the present disclosure is to provide an X-ray CT device, an image capture method, and a program that can clearly photograph a vein for each subject in CT angiography.

### Solution to Problem

In order to achieve the aforementioned objective, an X-ray CT device according to a first aspect of the present disclosure is an X-ray CT device capturing a CT image of a subject to whom a contrast agent has been administered by emitting an X-ray from an X-ray source toward the subject and detecting an X-ray transmitted through the subject with an X-ray detector, the X-ray CT device including:
an area setter setting, on the subject, a vein monitoring area that is an area for acquiring a CT value of a vein being monitored and a diagnostic area that is an area to be diagnosed of the subject;
a main scanner capturing a CT image of the diagnostic area at a timing when a contrast agent is present in an artery being monitored; and
a monitoring scanner monitoring whether a CT value in the vein monitoring area obtained by repeatedly emitting an X-ray from the X-ray source toward the subject is greater than or equal to a first threshold value after the main scanner finishes capturing a CT image of the diagnostic area,
wherein the main scanner captures a CT image of the diagnostic area when the monitoring scanner determines that a CT value in the vein monitoring area is greater than or equal to a first threshold value.

The monitoring scanner may cause the X-ray source to repeatedly emit a low-dose X-ray toward the vein monitoring area, and
the main scanner may cause the X-ray source to emit a high-dose X-ray that is higher in dose than the low-dose X-ray toward the diagnostic area.

The area setter may set, on the subject, an artery monitoring area that is an area for acquiring a CT value of the artery being monitored,
the monitoring scanner may monitor whether a CT value in the artery monitoring area obtained by repeatedly emitting an X-ray from the X-ray source toward the subject is greater than or equal to a second threshold value,
the main scanner may capture a CT image of the diagnostic area when the monitoring scanner determines that a CT value in the artery monitoring area is greater than or equal to a second threshold value, and
a cycle of X-ray emission to the vein monitoring area may be set longer than a cycle of X-ray emission to the artery monitoring area.

The area setter may set the vein monitoring area on a main portal vein of the subject, and set the diagnostic area to include a large intestine of the subject.

The monitoring scanner may monitor whether a CT value in the vein monitoring area is greater than or equal to a first threshold value after a pause time elapses from a point in time when the main scanner finishes capturing a CT image of the diagnostic area.

An image capture method according to a second aspect of the present disclosure is an image capture method capturing a CT image of a subject to whom a contrast agent has been administered by emitting an X-ray from an X-ray source toward the subject and detecting an X-ray transmitted through the subject with an X-ray detector, the image capture method including:
setting, on the subject, a vein monitoring area that is an area for acquiring a CT value of a vein being monitored and a diagnostic area that is an area to be diagnosed of the subject;
capturing a CT image of the diagnostic area at a timing when a contrast agent is present in an artery being monitored;
monitoring whether a CT value in the vein monitoring area obtained by repeatedly emitting an X-ray from the X-ray source toward the subject is greater than or equal to a first threshold value after finishing capturing a CT image of the diagnostic area; and
capturing a CT image of the diagnostic area when determining that a CT value in the vein monitoring area is greater than or equal to a first threshold value.

A program according to a third aspect of the present disclosure is a program for capturing a CT image of a subject to whom a contrast agent has been administered by emitting an X-ray from an X-ray source toward the subject and detecting an X-ray transmitted through the subject with an X-ray detector, the program causing a computer to function as:
area setting means for setting, on the subject, a vein monitoring area that is an area for acquiring a CT value of a vein being monitored and a diagnostic area that is an area to be diagnosed of the subject;
main scan means for capturing a CT image of the diagnostic area at a timing when a contrast agent is present in an artery being monitored; and
monitoring scan means for monitoring whether a CT value in the vein monitoring area obtained by repeatedly emitting an X-ray from the X-ray source toward the subject is greater than or equal to a first threshold value after the main scan means finishes capturing a CT image of the diagnostic area,
wherein the main scan means captures a CT image of the diagnostic area when the monitoring scan means determines that a CT value in the vein monitoring area is greater than or equal to a first threshold value.

### Advantageous Effects of Invention

According to the present disclosure, an X-ray CT device, an image capture method, and a program that can clearly photograph a vein for each subject in CT angiography can be provided.

### BRIEF DESCRIPTION OF DRAWINGS

FIG. 1 is a schematic diagram illustrating a configuration of an X-ray CT device according to the embodiment of the present disclosure;

FIG. 2 is a block diagram illustrating a hardware configuration of a processing unit according to the embodiment of the present disclosure;

FIG. 3A is a diagram illustrating one example of a data table of a scan condition storage according to the embodiment of the present disclosure;

FIG. 3B is a diagram illustrating one example of a data table of a monitoring condition storage according to the embodiment of the present disclosure;

FIG. 3C is a diagram illustrating one example of a data table of an image data storage according to the embodiment of the present disclosure;

FIG. 4 is a graph illustrating a monitoring area and a diagnostic area set by the X-ray CT device according to the embodiment of the present disclosure;

FIG. 5 is a diagram illustrating a procedure of X-ray emission executed by the X-ray CT device according to the embodiment of the present disclosure;

FIG. 6 is a graph illustrating an end condition for a monitoring scan by the X-ray CT device according to the embodiment of the present disclosure;

FIG. 7 is a flowchart illustrating a flow of image capture processing according to the embodiment of the present disclosure;

FIG. 8 is a flowchart illustrating a flow of monitoring scan processing according to the embodiment of the present disclosure;

FIG. 9 is a diagram illustrating a procedure of processing for a DBT method and a fixed delay time method in Example 1;

FIG. 10 is a diagram illustrating a CT image depicting measurement points of a subject in Example 1;

FIG. 11 is a diagram illustrating characteristics of a subject on which statistical processing is performed in Example 1;

FIG. 12 is a graph illustrating distribution of delay time in a case where the DBT method is applied in Example 1;

FIG. 13 is a diagram illustrating a CT value, a CNR, and an SNR of measurement points of a subject in Example 1;

FIG. 14A is a graph illustrating the number of images for each score obtained by image scoring of an observer A in Example 2;

FIG. 14B is a graph illustrating an average value of scores obtained by image scoring of the observer A in Example 2;

FIG. 15A is a graph illustrating the number of images for each score obtained by image scoring of an observer B in Example 2; and

FIG. 15B is a graph illustrating an average value of scores obtained by image scoring of the observer B in Example 2.

### DESCRIPTION OF EMBODIMENTS

Hereinafter, an X-ray CT device, an image capture method, and a program according to the embodiment of the present disclosure are described in detail with reference to drawings. In the drawings, the same or equivalent components are assigned with the same reference signs. In the embodiment, an orthogonal coordinate system is used in which a direction in which a rotation shaft of a gantry of the X-ray CT device extends is a Z-axis direction, a vertical direction is a Y-axis direction, and a direction extending on a horizontal plane orthogonal to a Y-axis and a Z-axis is an X-axis direction.

The X-ray CT device according to the embodiment is an image diagnostic device that captures a CT image (tomographic image) of a subject by using an X-ray. The X-ray CT device according to the embodiment may generate a two-dimensional CT image of a transverse-sectional slice of a subject, or may generate a three-dimensional CT image by arranging a plurality of two-dimensional CT images. The X-ray CT device according to the embodiment is configured capable of carrying out CT angiography on a subject. In CT angiography, X-ray photography is performed while injecting a contrast agent directly into a blood vessel of a subject by using a catheter, and a CT image depicting the blood vessel of the subject is captured. The contrast agent is administered to the subject to add contrast to the CT image or to emphasize a specific tissue. The contrast agent is, for example, an iodine contrast agent.

In CT angiography, an artery is photographed at a timing (arterial phase) when a contrast agent is estimated to reach the artery, and a vein is then photographed at a timing (venous phase) when the contrast agent is estimated to reach the vein. A bolus tracking (BT) method is publicly known as an approach to improve image quality of a CT image in the arterial phase. In the BT method, concentration of a contrast agent into an artery is monitored on a slice section set by a user, and a diagnostic area is photographed at a point in time when the concentration reaches target concentration.

In the BT method, normally, a fixed delay time is set between the arterial phase and the venous phase and a vein is photographed at a timing when the delay time elapses, but, as a result of diligent studies, the inventors have found that there is an individual difference in a timing when concentration of a contrast agent in a vein increases. In view of this, the X-ray CT device according to the embodiment is configured to optimize a photographing timing in the venous phase for each subject by monitoring concentration of a contrast agent in real time even in a case of photography in the venous phase.

The X-ray CT device according to the embodiment is suitable for, for example, CT angiography in which a diagnostic area is set on a large intestine and an area therearound. This CT angiography is preferably carried out before surgery of laparoscopically assisted right-sided colon resection and left-sided colon resection. This is because preoperative understanding of a position and a shape of a portal vein branch, for example, a gastro colic trunk, a middle colic vein, and an inferior mesenteric vein present around a large intestine is necessary.

Considering an anatomical structure of a large intestine and an area therearound, a downstream main portal vein is set as a vein being monitored in order to optimize a scan timing in consideration of hemodynamics of a portal vein branch for each subject. Regarding an artery, an aorta is set as an artery being monitored. Such an approach to monitor inflow of a contrast agent into an artery and a vein in real time in individually set areas is referred to as a double bolus tracking (DBT) method. Hereinafter, a case of photographing a diagnostic area of a subject by using the DBT method is described as an example.

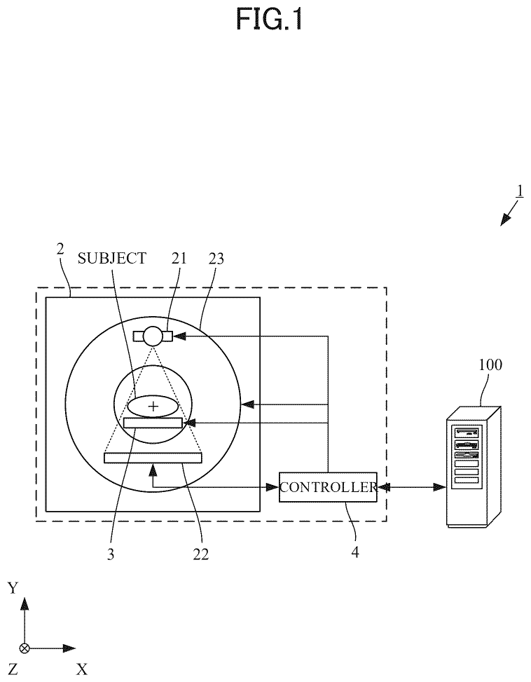

FIG. 1 is a schematic diagram illustrating a configuration of an X-ray CT device 1 according to the embodiment. The X-ray CT device 1 emits a low-dose X-ray toward a subject after a contrast agent is administered to the subject, and performs a monitoring scan on a vein being monitored to acquire a CT value that varies according to concentration of the contrast agent passing through the vein being monitored. Further, the X-ray CT device 1 emits a high-dose X-ray toward the subject and performs a main scan for capturing a CT image of a diagnostic area when the CT value of the vein being monitored becomes greater than or equal to a threshold value.

The X-ray CT device 1 includes a gantry 2, a bed 3, and a controller 4 installed in an examination room and a processing unit 100 installed in an operation room adjacent to the examination room. The gantry 2, the bed 3, and the controller 4 are one example of an image capture unit that captures a CT image of a monitoring area and a diagnostic area of a subject. The gantry 2 and the bed 3 are both installed on a floor surface of the examination room. The gantry 2 and the bed 3 are communicably connected to the processing unit 100 via a wired or wireless communication line and the controller 4.

The gantry 2 includes an X-ray source 21 that emits an X-ray toward a subject, an X-ray detector 22 that detects an X-ray emitted from the X-ray source 21 and attenuated as passing through the subject, and a rotating ring 23 that supports the X-ray source 21 and the X-ray detector 22. The X-ray source 21 and the X-ray detector 22 are supported by the rotating ring 23 in such a way as to face each other with the subject placed on the bed 3 therebetween, and the rotating ring 23 is supported rotatably about a rotation shaft (Z-axis) by a not-illustrated rotary drive mechanism.

The X-ray source 21 includes an X-ray tube that is one type of a vacuum tube and a high-voltage power supply that supplies voltage to the X-ray tube, and the X-ray tube generates an X-ray by the voltage from the high-voltage power supply. The X-ray detector 22 includes fine detection elements arranged in two dimensions, generates X-ray detection data by converting an X-ray detected with each detection element into an electric charge, and transmits the X-ray detection data to the processing unit 100. The X-ray detector 22 may be a single-slice type including a single row of detection elements in a slice direction (Z-axis direction), or may be a multi-slice type including a plurality of rows of detection elements in a slice direction.

The bed 3 includes a tabletop on which a subject is placed and a bed drive device that moves the tabletop in the Y-axis direction and the Z-axis direction and rotates the tabletop about the XYZ-axes. The bed 3 is introduced into a photographing port of the gantry 2 while the subject is placed thereon before X-ray emission by the gantry 2.

The controller 4 includes a memory and a processor, and, according to a program stored in a memory and a control signal supplied from the processing unit 100, the processor controls operations of the high-voltage power supply of the X-ray source 21, the X-ray detector 22, the rotary drive device of the rotating ring 23, and the bed drive device of the bed 3.

The processing unit 100 is, for example, a general-purpose computer. The processing unit 100 controls an operation of the controller 4, based on a program stored in a memory and an operation signal generated based on an operation of a user. Specifically, the processing unit 100 controls the controller 4 to move the X-ray source 21 and the X-ray detector 22 relatively to a subject by moving the bed 3 on which the subject is placed to a photographing position of the gantry 2 and rotating the rotating ring 23. Further, the processing unit 100 causes the X-ray source 21 to emit an X-ray, and causes the X-ray detector 22 to detect a passing X-ray passing through the subject and transmit detected X-ray detection data to the processing unit 100. The processing unit 100 reconstructs a CT image by performing rendering on the received X-ray detection data.

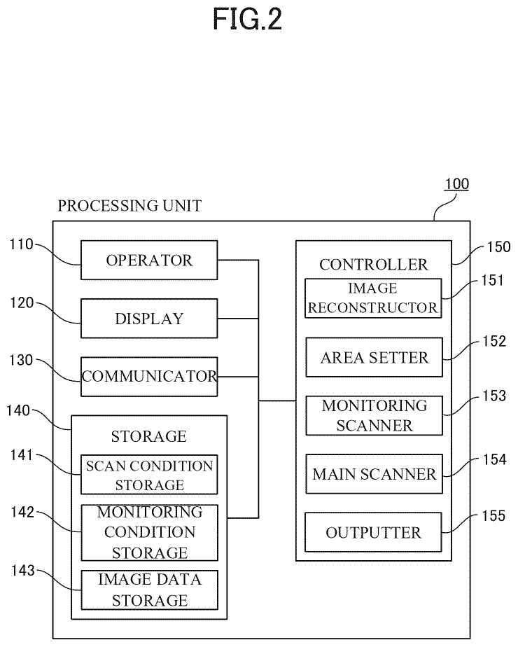

FIG. 2 is a block diagram illustrating a hardware configuration of the processing unit 100 according to the embodiment. The processing unit 100 includes an operator 110, a display 120, a communicator 130, a storage 140, and a controller 150. The components of the processing unit 100 are connected to one another via an internal bus.

The operator 110 accepts an instruction of a user, and supplies an operation signal corresponding to an accepted operation to the controller 150. The operator 110 includes, for example, a mouse and a keyboard.

The display 120 includes a display, and displays various types of images toward a user, based on image data supplied from the controller 150. The display 120 displays, for example, a CT image of a monitoring area and a diagnostic area.

The communicator 130 is a communication interface for the processing unit 100 to communicate with external equipment. The communicator 130 communicates with the controller 4 or other external equipment via, for example, a communication network such as the Internet or an input/output terminal such as a universal serial bus (USB).

The storage 140 includes, for example, a random access memory (RAM), a read only memory (ROM), a flash memory, and a hard disk. The storage 140 stores a program executed by the controller 150 or various types of data. Further, the storage 140 also functions as a work memory for temporarily storing various types of information and executing processing by the controller 150. Furthermore, the storage 140 includes a scan condition storage 141, a monitoring condition storage 142, and an image data storage 143.

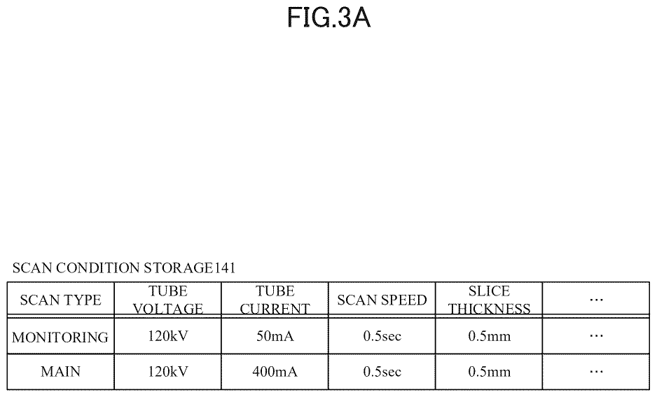

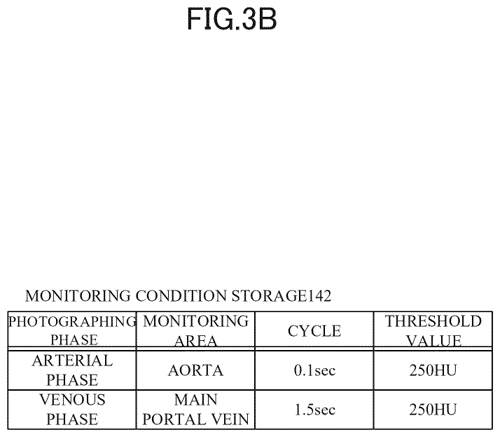

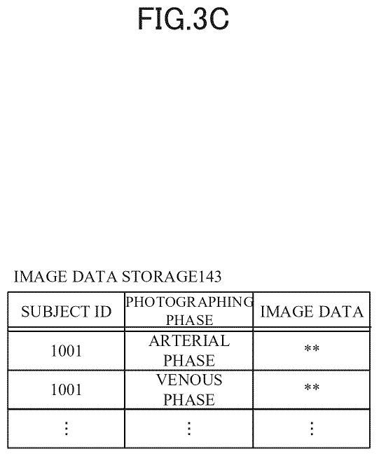

As illustrated in FIG. 3A, the scan condition storage 141 stores parameters that are set preliminarily by a user and are necessary for controlling scans toward a subject to be carried out sequentially by the gantry 2. The scans include a monitoring scan for monitoring a CT value of a monitoring area set in a subject and a main scan for capturing a CT image of a diagnostic area set in the subject. The parameters relating to scans are set for each scan type, and include a tube voltage, a tube current, a scan speed, a slice thickness for photography, a pause time, a start position, and an end position.

The tube voltage and the tube current are a voltage value and a current value of current supplied from the high-voltage power supply to the X-ray source 21. The scan speed is time required for the rotating ring 23 to rotate once. The photographing slice thickness is an interval in a slice direction (Z-axis direction) of a two-dimensional CT image generated by a scan. The pause time is time from a point in time when a scan is available to a point in time when the scan is started. The start position and the end position are positions in the Z-axis direction of the gantry 2 at a time of scan start and a time of scan end toward a subject placed on the bed 3.

As illustrated in FIG. 3B, the monitoring condition storage 142 stores parameters that are set preliminarily by a user and are necessary for controlling monitoring scans for a subject. The parameters relating to monitoring are set for each photographing phase, and include a monitoring area, a cycle of X-ray emission, and a threshold value for a CT value of the monitoring area.

The photographing phase is any of the arterial phase and the venous phase. The monitoring area is an area for which a CT value is acquired from a CT image obtained by a monitoring scan. The monitoring area is set on an artery and a vein being monitored. The cycle of X-ray emission is a cycle of X-ray emission repeatedly performed on a subject during a monitoring scan.

As illustrated in FIG. 3C, the image data storage 143 stores data of a captured CT image of a diagnostic area, in association with a subject identification (ID) assigned to each subject and the photographing phase.

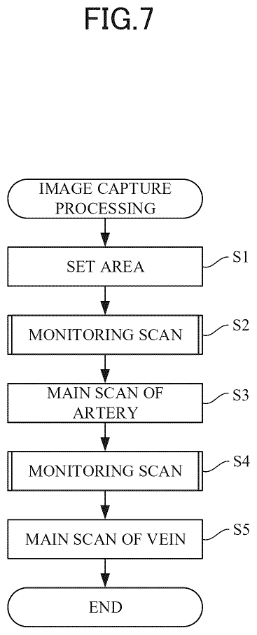

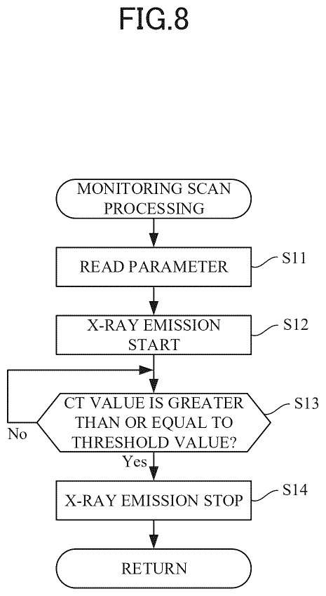

Returning to FIG. 2, the controller 150 includes a processor, and controls the components of the processing unit 100. The processor is, for example, a central processing unit (CPU). The controller 150 includes an internal timer for counting time. Further, the controller 150 executes image capture processing in FIG. 7 and monitoring scan processing in FIG. 8 by executing a program stored in the storage 140. The controller 150 functionally includes an image reconstructor 151, an area setter 152, a monitoring scanner 153, a main scanner 154, and an outputter 155.

The image reconstructor 151 acquires X-ray detection data transmitted from the X-ray detector 22, and reconstructs a CT image of a subject by performing rendering based on the acquired X-ray detection data. For image reconstruction, for example, a convolutional back projection method is used.

The area setter 152 sets, on a subject, an artery monitoring area for acquiring a CT value of an artery being monitored, a vein monitoring area for acquiring a CT value of a vein being monitored, and a diagnostic area that is an area to be image-diagnosed, based on an operation signal from the operator 110 accepting an operation of a user. Specifically, the area setter 152 causes the X-ray source 21 to emit an X-ray while the bed 3 on which a subject is placed is positioned relative to the gantry 2, causes the image reconstructor 151 to generate a CT image based on detection data detected by the X-ray detector 22, and causes the display 120 to display the CT image on the display. A user sets an artery monitoring area, a vein monitoring area, and a diagnostic area by operating the operator 110 with reference to the CT image displayed on the display.

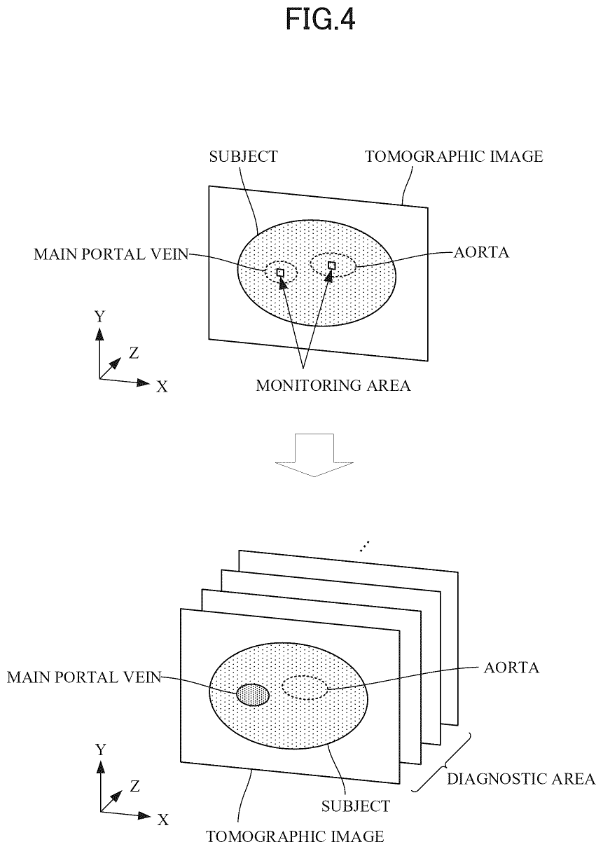

As illustrated in FIG. 4, the artery monitoring area is a monitoring area set on an artery being monitored, and is set on, for example, an aorta. The vein monitoring area is a monitoring area set on a vein being monitored, and is set on, for example, a main portal vein. The diagnostic area is set as to include, for example, a large intestine.

The monitoring areas are preferably set on an identical transverse section (XY plane) of a subject. In particular, the monitoring areas are preferably set on a transverse section including a thoracic diaphragm. Further, the monitoring areas may be a single pixel, or may be set to include a plurality of pixels. When the monitoring area is a single pixel, the monitoring area is expressed by XYZ-coordinate values of the pixel, and when the monitoring area is configured by a plurality of pixels, the monitoring area is expressed by maximum coordinates and minimum coordinates of the XYZ-axes.

The diagnostic area is expressed by, for example, a Z-coordinate value at a time of scan start and a time of scan end. The diagnostic area may include at least one of the artery monitoring area and the vein monitoring area, or may include none of the artery monitoring area and the vein monitoring area.

Returning to FIG. 2, the monitoring scanner 153 executes a monitoring scan for monitoring whether a CT value in the artery monitoring area set by the area setter 152 is greater than or equal to a threshold value (second threshold value) for the arterial phase stored in the monitoring condition storage 142.

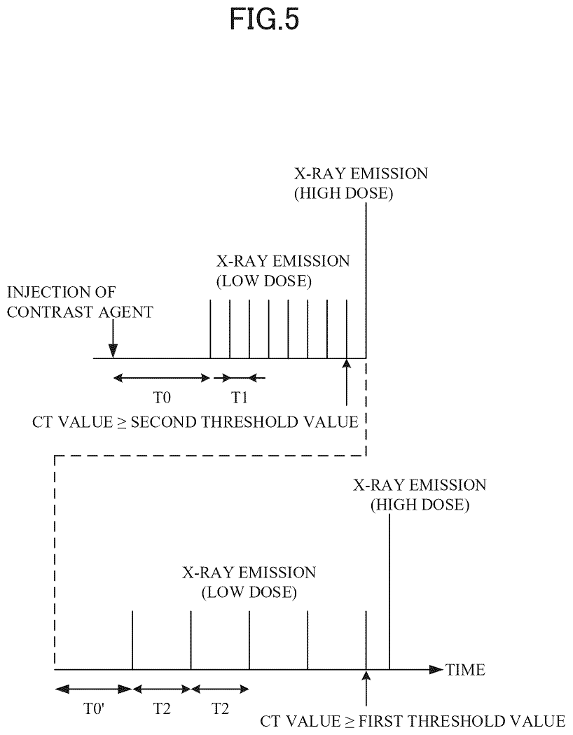

To be more specific, as illustrated in FIG. 5, the monitoring scanner 153 causes the X-ray source 21 to start cyclic low-dose X-ray emission at a cycle of T1 toward a subject after a pause time TO elapses from a point in time when a contrast agent is injected. The pause time TO is set in advance by a user in consideration of a timing at which the contrast agent reaches an artery being monitored, and is stored in the scan condition storage 141. Use of the low-dose X-ray is for a purpose of reducing radiation exposure of a subject caused by a monitoring scan.

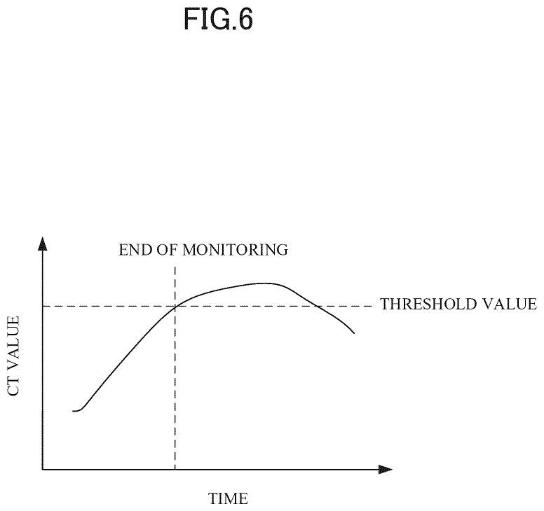

The monitoring scanner 153 causes the image reconstructor 151 to repeatedly generate a CT image based on X-ray detection data detected by the X-ray detector 22, and cyclically acquires a CT value of the artery monitoring area from the generated CT image. At this time, a CT value of an artery being monitored gradually increases as concentration of the contrast agent in the artery increases, as illustrated in FIG. 6. The acquired CT value is sequentially compared with the second threshold value, and when the CT value is determined as becoming greater than or equal to the second threshold value, a monitoring scan with a low-dose X-ray is stopped.

Further, the monitoring scanner 153 executes a monitoring scan for monitoring whether a CT value in the vein monitoring area set by the area setter 152 is greater than or equal to a threshold value (first threshold value) for the venous phase stored in the monitoring condition storage 142 after the main scanner 154 finishes photographing the diagnostic area.

To be more specific, as illustrated in FIG. 5, the monitoring scanner 153 causes the X-ray source 21 to start cyclic low-dose X-ray emission at a cycle of T2 toward a subject after a pause time T0′ elapses from a point in time when photography in the arterial phase is finished. The pause time T0′ is set in advance by a user in consideration of a timing at which the contrast agent reaches a vein being monitored, and is stored in the scan condition storage 141. The pause time T0′ may be 0. The cycle T2 is set longer than the cycle T1. This is because venous blood flow is slower than arterial blood flow.

The monitoring scanner 153 causes the image reconstructor 151 to repeatedly generate a CT image based on X-ray detection data detected by the X-ray detector 22, and cyclically acquires a CT value of the artery monitoring area from the CT image. At this time, a CT value of an artery being monitored gradually increases as concentration of the contrast agent in the artery increases, as illustrated in FIG. 6. The acquired CT value is sequentially compared with the first threshold value, and when the CT value is determined as becoming greater than or equal to the first threshold value, a monitoring scan with a low-dose X-ray is stopped.

Note that, when the monitoring area is set to include a plurality of pixels in any monitoring scan, for example, a monitoring end condition is determined as satisfied when a CT value of any of the pixels is greater than or equal to the threshold value or when an average CT value of the pixels is greater than or equal to the threshold value.

Returning to FIG. 2, the main scanner 154 executes a main scan for photographing the diagnostic area set by the area setter 152 when the monitoring scanner 153 determines that the CT value in the artery monitoring area is greater than or equal to the second threshold value and when the monitoring scanner 153 determines that the CT value in the vein monitoring area is greater than or equal to the first threshold value. Specifically, the main scanner 154 causes the X-ray source 21 to emit an X-ray higher in dose than in a monitoring scan toward a subject, and causes the image reconstructor 151 to generate a CT image based on X-ray detection data detected by the X-ray detector 22. Next, the main scanner 154 causes the image data storage 143 to store the CT image in association with the subject ID and the photographing phase.

The outputter 155 outputs a CT image generated by the image reconstructor 151 to outside. The outputter 155 transmits, for example, data of a CT image to the display 120, and causes the display 120 to display the CT image on the display.

The above is the configuration of the processing unit 100.

### Image Capture Processing

Hereinafter, a flow of image capture processing executed by the processing unit 100 is described with reference to a flowchart in FIG. 7. The image capture processing is processing of capturing a CT image of a diagnostic area of a subject in the arterial phase and the venous phase. The image capture processing is started at a point in time when the bed 3 on which a subject is placed is positioned relative to the gantry 2 and a user operates the operator 110 to instruct start of the image capture processing.

First, the area setter 152 sets an artery monitoring area, a vein monitoring area, and a diagnostic area, based on an operation signal from the operator 110 accepting an operation of the user (Step S1). The user operates the operator 110 to set the artery monitoring area on an aorta of the subject, the vein monitoring area on a main portal vein of the subject, and the diagnostic area to include a large intestine of the subject, as illustrated in FIG. 4. In this setting, the user inputs, to the operator 110, XYZ-coordinate values defining the monitoring area and a Z-coordinate value defining the diagnostic area. The area setter 152 causes the monitoring condition storage 142 in FIG. 3A and the scan condition storage 141 in FIG. 3B to store parameters accepted by the operator 110.

When finishing inputting the monitoring area and the diagnostic area, the user administers a contrast agent to a blood vessel of the subject. When the operator 110 accepts an input that the contrast agent has been administered from the user, the monitoring scanner 153 counts a pause time T0 set in advance by using an internal timer, and executes a monitoring scan for monitoring whether a CT value in the artery monitoring area set in the processing in Step S1 is greater than or equal to a second threshold value after the count ends (Step S2). Hereinafter, a flow of monitoring scan processing executed by the processing unit 100 according to the embodiment is described with reference to FIG. 8.

### Monitoring Scan Processing

First, the monitoring scanner 153 reads various types of parameters from the scan condition storage 141 in FIG. 3A and the monitoring condition storage 142 in FIG. 3B (Step S11). Specifically, the monitoring scanner 153 reads parameters relating to a monitoring scan from the scan condition storage 141 in FIG. 3A, and reads parameters relating to the arterial phase from the monitoring condition storage 142 in FIG. 3B.

Next, the monitoring scanner 153 starts cyclic X-ray emission from the X-ray source 21 toward the subject, based on the parameters read in the processing in Step S11 (Step S12). The monitoring scanner 153 repeats processing of cyclically emitting a low-dose X-ray from the X-ray source 21 toward the subject, causing the image reconstructor 151 to generate a CT image based on X-ray detection data detected by the X-ray detector 22, and acquiring a CT value in the artery monitoring area of the generated CT image.

Next, the monitoring scanner 153 determines whether the CT value acquired in the processing in Step S12 is greater than or equal to a second threshold value (Step S13). The second threshold value is a threshold value corresponding to the arterial phase stored in the monitoring condition storage 142 in FIG. 3B. As concentration of the contrast agent in the artery increases, the CT value in the artery monitoring area also increases, as illustrated in FIG. 6.

When the acquired CT value is determined as greater than or equal to the second threshold value (Step S13; Yes), the monitoring scanner 153 stops the cyclic X-ray emission from the X-ray source 21 toward the subject (Step S14), and returns the processing. On the other hand, when the acquired CT value is determined as less than the second threshold value (Step S13; No), the monitoring scanner 153 continues the cyclic X-ray emission from the X-ray source 21 toward the subject until the CT value becomes greater than or equal to the second threshold value.

The above is the flow of the monitoring scan processing.

Returning to FIG. 7, after the processing in Step S2 ends, the main scanner 154 executes a main scan for photographing the diagnostic area set by the area setter 152 while the contrast agent is present in the artery being monitored (Step S3). Specifically, the main scanner 154 reads parameters relating to a main scan from the scan condition storage 141 in FIG. 3A. Next, the main scanner 154 causes the X-ray source 21 to emit a high-dose X-ray toward the subject, based on the read parameters. Next, the main scanner 154 causes the image reconstructor 151 to generate a CT image based on X-ray detection data detected by the X-ray detector 22, and causes the image data storage 143 in FIG. 3C to store the CT image in association with the subject ID and the arterial phase.

Next, the monitoring scanner 153 executes a monitoring scan for monitoring whether a CT value in the vein monitoring area set by the area setter 152 is greater than or equal to a first threshold value after waiting the processing for a pause time T0′ (Step S4). A specific flow of processing is as illustrated in FIG. 8, similarly to the processing in Step S2. The cycle of X-ray emission is T2, and the first threshold value is a threshold value corresponding to the venous phase stored in the monitoring condition storage 142 in FIG. 3B.

After the processing in Step S4 ends, the main scanner 154 executes a main scan for photographing the diagnostic area set by the area setter 152 while the contrast agent is present in the vein being monitored (Step S5), and ends the processing. A specific flow of processing is the same or equivalent to the processing in Step S3.

The above is the flow of the image capture processing.

As described above, the X-ray CT device 1 according to the embodiment includes: the main scanner 154 that captures a CT image of a diagnostic area at a timing when a contrast agent is present in an artery being monitored; and the monitoring scanner 153 that monitors whether a CT value in a vein monitoring area obtained by repeatedly emitting an X-ray toward a subject is greater than or equal to a first threshold value after the main scanner 154 finishes photographing the diagnostic area, and the main scanner 154 is configured to photograph the diagnostic area when the monitoring scanner 153 determines that the CT value in the vein monitoring area is greater than or equal to the first threshold value. This enables clear photography of a vein for each subject in X-ray photography using a contrast agent.

The present disclosure is not limited to the above embodiment, but may be modified as follows.

### Modification

In the above embodiment, a diagnostic area is set on a three-dimensional area and captured CT images are overlaid to generate a three-dimensional CT image, but the present disclosure is not limited thereto. For example, a diagnostic area may be set on a two-dimensional area on an XY plane. At this time, the diagnostic area is expressed by a position in the Z-axis direction.

In the above embodiment, a monitoring area and a diagnostic area are set by accepting an operation of a user by the operator 110, but the present disclosure is not limited thereto. For example, the present disclosure may be configured in such a way that, when a user operates the operator 110 to set an artery being monitored, a vein, and a part to be diagnosed, the processing unit 100 recognizes the artery being monitored, the vein, and the part to be diagnosed in a CT image captured with reference to a prediction model stored in the storage 140 and sets a monitoring area and a diagnostic area on the recognized locations.

In the above embodiment, X-ray dose for a main scan is set higher than X-ray dose for a monitoring scan, but the present disclosure is not limited thereto. For example, dose for a monitoring scan may be set the same or equivalent to a main scan.

In the above embodiment, the cycle T1 of X-ray emission in the arterial phase is set smaller than the cycle T2 of X-ray emission in the venous phase, but the present disclosure is not limited thereto. For example, the cycles T1 and T2 may be set the same or equivalent.

In the above embodiment, the cycle of X-ray emission for a monitoring scan is fixed in each of the arterial phase and the venous phase, but the present disclosure is not limited thereto. For example, the cycle of X-ray emission may vary regularly within a set range, or may vary randomly.

In the above embodiment, a monitoring area is set preliminarily and is stored in the monitoring condition storage 142, but the present disclosure is not limited thereto. For example, the X-ray CT device 1 may be configured to cause the display 120 to display a CT image acquired by a monitoring scan on a display in real time, measure a CT value in a part of the CT image on which a cursor is placed, and display the CT value. In the above configuration, when a user operates the operator 110 to place a cursor on a monitoring area, a CT value corresponding to the cursor may be displayed on the display. Then, when the user refers to the CT value displayed on the display and determines that the CT value is greater than or equal to a threshold value, the operator 110 may be operated to execute a main scan.

In the above embodiment, the processing unit 100 executes a main scan without a standby time when a CT value of a monitoring area is greater than or equal to a threshold value, but the present disclosure is not limited thereto. The processing unit 100 may carry out a main scan after a standby time set in advance elapses from a point in time when a CT value of a monitoring area becomes greater than or equal to a threshold value.

In the above embodiment, the processing unit 100 carries out a monitoring scan in both of the arterial phase and the venous move, but the present disclosure is not limited thereto. For example, the processing unit 100 may be configured to perform a monitoring scan in the venous phase and carry out a main scan in the arterial phase at a point in time when a delay time set in advance elapses from administration of a contrast agent.

In the above embodiment, parameters relating to scans are stored in the scan condition storage 141 and parameters relating to monitoring are stored in the monitoring condition storage 142, but the present disclosure is not limited thereto. For example, parameters relating to scans and monitoring may be stored in an identical data table in order of execution of a monitoring scan and a main scan.

In the above embodiment, various types of data are stored in the storage 140 of the processing unit 100, but the present disclosure is not limited thereto. For example, the whole or a part of various types of data may be stored in an external control device or a processing unit via a communication network.

In the above embodiment, the processing unit 100 operates based on a program stored in the storage 140, but the present disclosure is not limited thereto. For example, a functional configuration achieved by a program may be achieved by hardware.

In the above embodiment, the processing unit 100 is, for example, a general-purpose computer, but the present disclosure is not limited thereto. For example, the processing unit 100 may be achieved by a processing unit on a cloud.

In the above embodiment, processing executed by the processing unit 100 is achieved by a device including the above-described physical configuration executing a program stored in the storage 140, but the present disclosure may be achieved as a program, or may be achieved as a storage medium on which the program is recorded.

Further, a device may be configured that executes the above-described processing operation by installing, on a processing unit, a program for executing the above-described processing operation, the program being stored and distributed in a non-transitory recording medium readable by a processing unit, such as a flexible disk, a compact disk read-only memory (CD-ROM), a digital versatile disk (DVD), or a magneto-optical disk (MO).

In the above embodiment, a diagnostic area is set on a large intestine and an area therearound, but the present disclosure is not limited thereto. When a vein monitoring area is set on a main portal vein, a diagnostic area may be set to include, for example, a liver, a gallbladder, a pancreas, a spleen, or a kidney. Further, a vein monitoring area is not limited to a main portal vein, but may be set on, for example, a superior vena cava, an inferior vena cava, a pulmonary vein, a subclavian vein, or a femoral vein.

The above embodiment is illustrative, and the present disclosure is not limited thereto, but various embodiments can be made within a scope not departing from the gist of the disclosure described in claims. The components described in the embodiment or the modification can be feely combined. Further, equivalents to the disclosure described in claims are also included in the present disclosure. In addition, even when the components of the disclosure described in claims have the same names as the components described in the above embodiment, the components of the disclosure are not limited to the components per se described in the above embodiment, but can be modified and applied as appropriate.

Hereinafter, the present disclosure is specifically described by using examples. However, the present disclosure is not limited to the examples.

### Example 1

In Example 1, preoperative CT angiography (CTA) was carried out on a main portal vein by applying the DBT method of individualizing a scan timing in the venous phase for each subject to test whether an improved contrast effect on a gastro colic trunk, a middle colic vein, and an inferior mesenteric vein and improved precision of a vein in a CT image can be achieved. As a comparative example, preoperative CTA was carried out using a fixed delay time method. The test subjects were 68 patients undergoing preoperative CTA for colorectal cancer. The patients were divided into two groups of a DBT group (n=35) and a fixed delay time group (n=33).

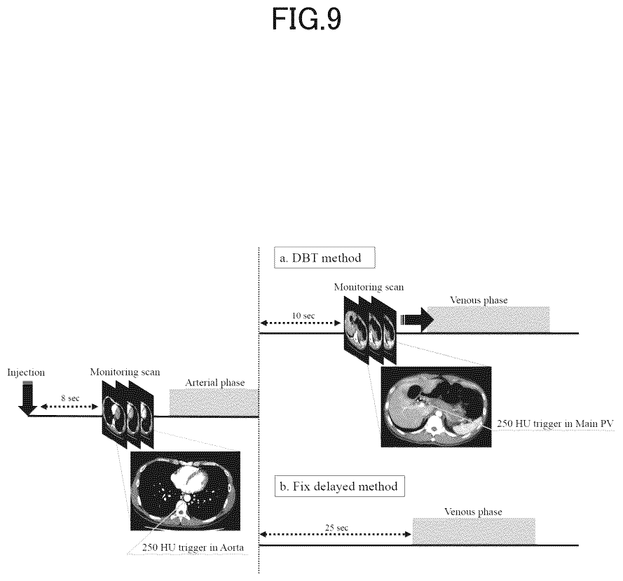

The procedures for the DBT method and the fixed delay time method are as illustrated in FIG. 9. To be more specific, first, a medical professional entered a venous catheter through a brachial vein and injected a contrast agent. Next, a photography start timing for the arterial phase was determined using a bolus-tracking protocol of an X-ray CT device. Specifically, a low-dose monitoring scan was performed at a position of a diaphragmatic dome, and a CT image in the arterial phase was acquired at a point in time when a region of interest (ROI) in an abdominal aorta reached a threshold value 250 HU. Next, in the DBT group, a low-dose monitoring scan was performed at a hepatic portal level at an interval of 1.5 seconds, and a CT image in the venous phase was acquired at a point in time when ROIs in the main portal vein reached 250 HU. On the other hand, in the fixed delay time group, a CT image in the venous phase was acquired with a delay time of 25 seconds after a point in time when the CT image in the arterial phase was acquired. As the X-ray CT device, an 80-row CT system (manufactured by Canon Medical Systems, Corp.) was used.

All CT images obtained by the CT system were reconstructed with three sections (axial, coronal, and sagittal) in a 2.0 mm slice thickness by iterative approximation applied reconstruction using a reconstruction function for abdomen, and were subjected to 3D volume rendering executed in a work station.

As estimation values of radiation exposure in a low-dose monitoring, a CT dose index (CTDI_{VOI}) and a dose length product (DLP) were used. The CTDI_{VOI} and is a value obtained by dividing a dose length product that is a dose profile obtained by a single scan by an X-ray beam width. The DLP is a product of the CTDI_{VOI} and a length L of an image capture range when a helical scan is performed within the image capture range with the length L. Both of the parameters were acquired from a picture archiving and communication system (PACS) that is connected to the X-ray CT device and to which scan data are automatically reported.

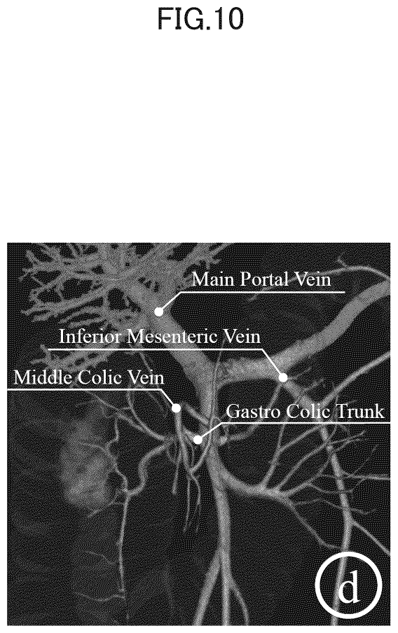

The quantitative evaluation of a CT image was performed by a blinded reader. As illustrated in FIG. 10, a ROI (~10 mm²) was set on blood vessel roots of a main portal vein (MPV), a gastro colic trunk (GCT), a middle colic vein (MCV), and an inferior mesenteric vein (IMV), a ROI (size ~5 cm²) was set on an erector spinae muscle at a height of an umbilicus, and an average CT value was measured. A contrast-to-noise ratio (CNR) and a signal-to-noise ratio (SNR) were calculated based on the measured average CT value. The CNR and the SNR are expressed by following equations.

$${CNR}_{ROI} = ( R O {I}_{ROI} - RO {I}_{muscle} ) / {SD}_{fat}$$

$${SNR}_{ROI} = {ROI}_{ROI} / {SD}_{fat}$$

Herein, ROI_{muscle} is an average attenuation of the erector spinae muscle, and SD_{fat} is a standard deviation of pixel values in uniform abdominal wall fat in an anterior abdominal wall.

Statistical processing was performed on each piece of acquired data. The chi-square test was used to test for gender differences, and the student-t test was used to compare patient characteristics and radiation dose estimation values. Further, Mann-Whitney's U test was used to compare quantitative data between the two groups. A P-value was two-tailed, and P<0.01 was defined as indicative of a statistically significant difference.

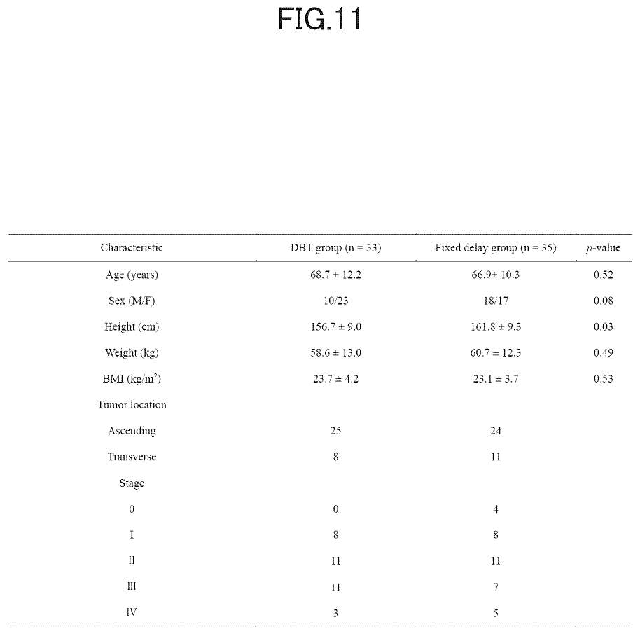

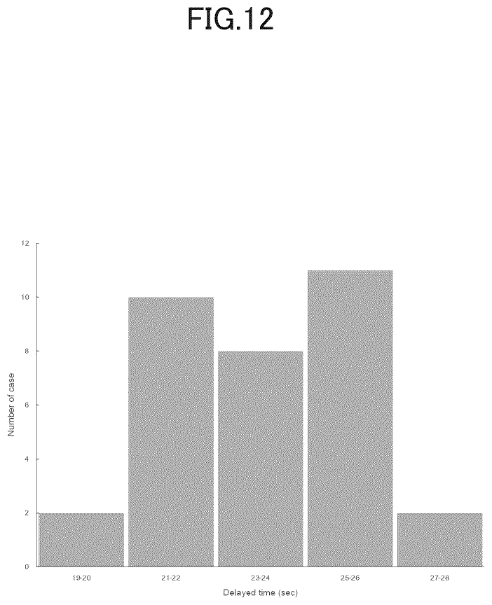

Results of the experiment are indicated below. The patient characteristics such as age, height, weight and BMI were not significantly different between the two groups, as illustrated in FIG. 11. The average number of monitoring scans in the DBT group was 3 (range: 1 to 6). The average delay time from the arterial phase to the venous phase in the DBT group was widely distributed as illustrated in FIG. 12, with an average of 24.0-2.1 seconds (range: 20.4 to 27.6 seconds). Further, the average CTDI_{VOI} for low-dose monitoring was 7.7±3.0 mGy, and the DLP was 15.5±6.0 mGy·cm.

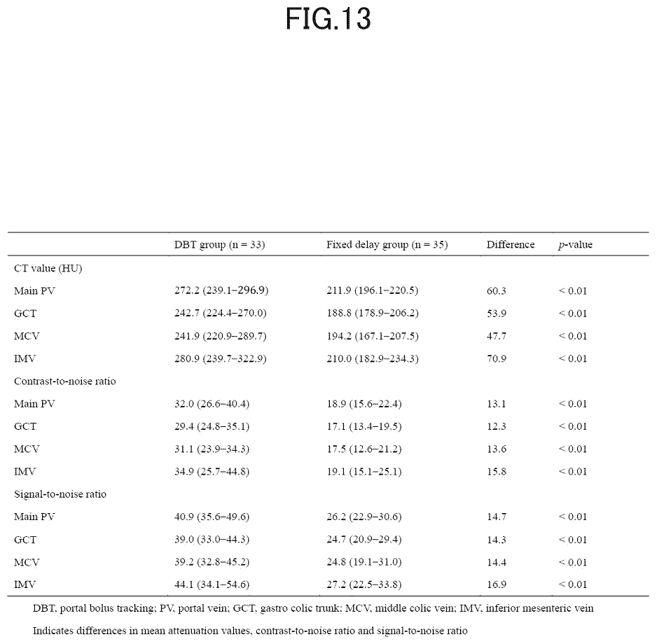

The average CT values of the blood vessel roots of the main portal vein, the gastro colic trunk, the middle colic vein, and the inferior mesenteric vein in each group are as illustrated in FIG. 13. The average CT values of all portal vein branches were higher in the DBT group, indicating an improved contrast effect on the veins compared to the fixed delay time method. The largest difference between the two groups was observed in the inferior mesenteric vein. Further, both CNR_{ROI} and SNR_{ROI} were significantly higher in the DBT group.

### Example 2

In Example 2, CTA for photographing a large intestine of a subject was carried out by using the DBT method and the fixed delay time method similarly to Example 1, and then, two blinded surgeons carried out image quality scoring on a three-dimensional CT image obtained by CTA on a 5-point scale below.
1 Non-diagnostic; Gastro colic trunk not depicted
2 Poor; Only root of gastro colic trunk depicted
3 Moderate; Distal part of gastro colic trunk depicted
4 Good; Peripheral branch of gastro colic trunk depicted
5 Excellent; Peripheral branch of gastro colic trunk clearly depicted

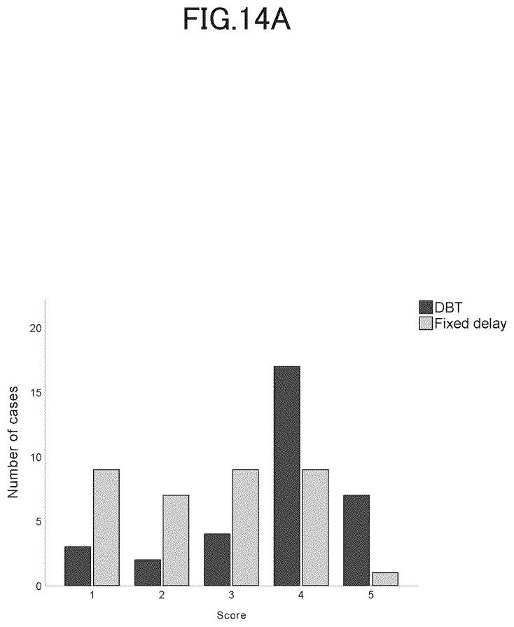

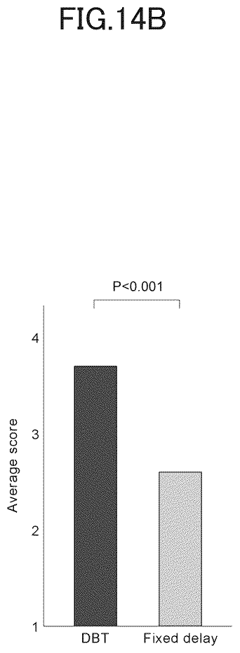

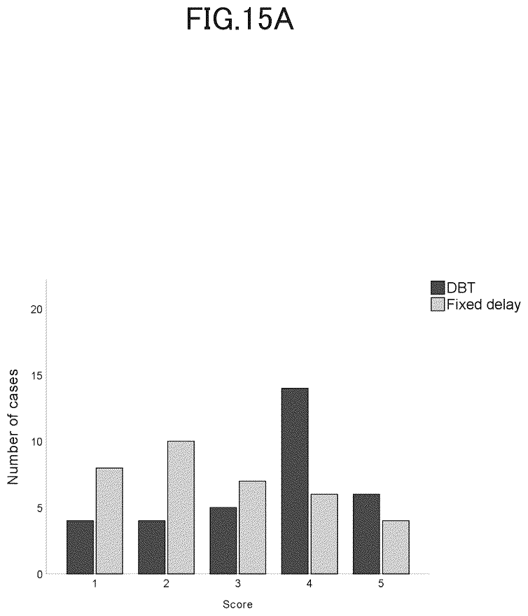

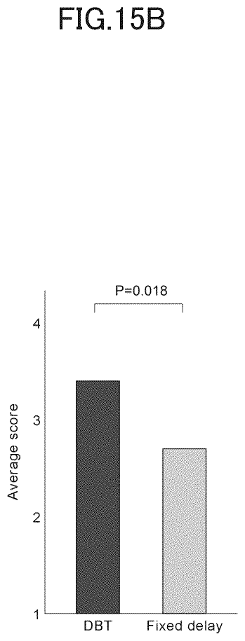

Hereinafter, evaluation results are indicated with reference to FIGS. 14A and B and FIGS. 15A and B. FIGS. 14A and B illustrate evaluation results by an observer A, and FIGS. 15A and B illustrate evaluation results by an observer B. Graphs in FIGS. 14A and 15A indicate the number of images for each score in each of the DBT method and the fixed delay time method, and graphs in FIGS. 14A and 15B indicate an average value of scores. As can be understood from the graphs, few cases were evaluated as non-diagnostic or poor, by both the observers A and B, in the DBT method compared to the fixed delay time method, and many cases were evaluated as depicting or clearly depicting the peripheral branch of the gastrointestinal trunk. From the above, the DBT method was confirmed as photographing the veins more clearly than the fixed delay time method.

This application claims the benefit of Japanese Patent Application No. 2022-199377, filed on Dec. 14, 2022, the entire disclosure of which is incorporated by reference herein.

### INDUSTRIAL APPLICABILITY

The X-ray CT device, the image capture method, and the program according to the present disclosure enable clear photography of a vein for each subject in CT angiography, and are thus useful.

### REFERENCE SIGNS LIST

1 X-ray CT device
2 Gantry
3 Bed
4 Controller
21 X-ray source
22 X-ray detector
23 Rotating ring
100 Processing unit
110 Operator
120 Display
130 Communicator
140 Storage
141 Scan condition storage
142 Monitoring condition storage
143 Image data storage
150 Controller
151 Image reconstructor
152 Area setter
153 Monitoring scanner
154 Main scanner
155 Outputter

1. An X-ray CT device capturing a CT image of a subject to whom a contrast agent has been administered by emitting an X-ray from an X-ray source toward the subject and detecting an X-ray transmitted through the subject with an X-ray detector, the X-ray CT device comprising:
an area setter setting, on the subject, a vein monitoring area that is an area for acquiring a CT value of a vein being monitored and a diagnostic area that is an area to be diagnosed of the subject;
a main scanner capturing a CT image of the diagnostic area at a timing when a contrast agent is present in an artery being monitored; and
a monitoring scanner monitoring whether a CT value in the vein monitoring area obtained by repeatedly emitting an X-ray from the X-ray source toward the subject is greater than or equal to a first threshold value after the main scanner finishes capturing a CT image of the diagnostic area,
wherein the main scanner captures a CT image of the diagnostic area when the monitoring scanner determines that a CT value in the vein monitoring area is greater than or equal to a first threshold value.

2. The X-ray CT device according to claim 1, wherein the monitoring scanner causes the X-ray source to repeatedly emit a low-dose X-ray toward the vein monitoring area, and
the main scanner causes the X-ray source to emit a high-dose X-ray that is higher in dose than the low-dose X-ray toward the diagnostic area.

3. The X-ray CT device according to claim 1, wherein
the area setter sets, on the subject, an artery monitoring area that is an area for acquiring a CT value of the artery being monitored,
the monitoring scanner monitors whether a CT value in the artery monitoring area obtained by repeatedly emitting an X-ray from the X-ray source toward the subject is greater than or equal to a second threshold value,
the main scanner captures a CT image of the diagnostic area when the monitoring scanner determines that a CT value in the artery monitoring area is greater than or equal to a second threshold value, and
a cycle of X-ray emission to the vein monitoring area is set longer than a cycle of X-ray emission to the artery monitoring area.

4. The X-ray CT device according to claim 1 the 3, wherein the area setter sets the vein monitoring area on a main portal vein of the subject, and sets the diagnostic area to include a large intestine of the subject.

5. The X-ray CT device according to claim 1, wherein the monitoring scanner monitors whether a CT value in the vein monitoring area is greater than or equal to a first threshold value after a pause time elapses from a point in time when the main scanner finishes capturing a CT image of the diagnostic area.

6. An image capture method capturing a CT image of a subject to whom a contrast agent has been administered by emitting an X-ray from an X-ray source toward the subject and detecting an X-ray transmitted through the subject with an X-ray detector, the image capture method comprising:
setting, on the subject, a vein monitoring area that is an area for acquiring a CT value of a vein being monitored and a diagnostic area that is an area to be diagnosed of the subject;
capturing a CT image of the diagnostic area at a timing when a contrast agent is present in an artery being monitored;
monitoring whether a CT value in the vein monitoring area obtained by repeatedly emitting an X-ray from the X-ray source toward the subject is greater than or equal to a first threshold value after finishing capturing a CT image of the diagnostic area; and
capturing a CT image of the diagnostic area when determining that a CT value in the vein monitoring area is greater than or equal to a first threshold value.

7. A program for capturing a CT image of a subject to whom a contrast agent has been administered by emitting an X-ray from an X-ray source toward the subject and detecting an X-ray transmitted through the subject with an X-ray detector, the program causing a computer to function as:
area setting means for setting, on the subject, a vein monitoring area that is an area for acquiring a CT value of a vein being monitored and a diagnostic area that is an area to be diagnosed of the subject;
main scan means for capturing a CT image of the diagnostic area at a timing when a contrast agent is present in an artery being monitored; and
monitoring scan means for monitoring whether a CT value in the vein monitoring area obtained by repeatedly emitting an X-ray from the X-ray source toward the subject is greater than or equal to a first threshold value after the main scan means finishes capturing a CT image of the diagnostic area,
wherein the main scan means captures a CT image of the diagnostic area when the monitoring scan means determines that a CT value in the vein monitoring area is greater than or equal to a first threshold value.
